(19) United States
(12) Patent Application Publication
(10) Pub. No.: US 2023/0125178 A1
(43) Pub. Date: Apr. 27, 2023

(54) TRANSPOSABLE INTRAOCULAR LENS

(71) Applicant: Alcon Inc., Fribourg (CH)

(72) Inventor: George Hunter PETTIT, Fort Worth, TX (US)

(21) Appl. No.: 18/050,045

(22) Filed: Oct. 26, 2022

Related U.S. Application Data
(60) Provisional application No. 63/263,141, filed on Oct. 27, 2021.

(51) Int. Cl. A61F 2/16

(52) U.S. Cl. CPC A61F 2/1627; A61F 2/164; A61F 2/1648

(57) ABSTRACT

Certain aspects of the present disclosure provide a transposable intraocular lens (IOL), which includes a lens body, including a first lens portion having a first outer surface with a first radius of curvature, a second lens portion having a second outer surface with a second radius of curvature that is different from the first radius of curvature, and a central optic portion between the first lens portion and the second lens portion, and a haptic portion that is coupled to the lens body. The transposable IOL also includes a haptic portion configured to support the transposable IOL whether in a first orientation of implantation in a patient’s eye or in a transposed second orientation of implantation in the patient’s eye.

### PRIORITY CLAIM

This application claims the benefit of priority of U.S. Provisional Pat. Application Serial No. 63/263,141 titled “TRANSPOSABLE INTRAOCULAR LENS,” filed on Oct. 27, 2021, whose inventor is George Hunter Pettit, which is hereby incorporated by reference in its entirety as though fully and completely set forth herein.

### BACKGROUND

Cataract surgery involves removing a cataractous lens of a patient’s eye and replacing the lens with an artificial intraocular lens (IOL). Planning for cataract surgery typically involves selecting an IOL with an IOL power that is able to achieve a desired refractive outcome or target post-surgery. The determination of an IOL power necessary to achieve a particular post-operative refraction outcome is dependent on measurements of the anatomical parameters of the patient’s eye, such as one or more of the axial length of the eye, corneal curvature, anterior chamber depth, white-to-white diameter of the cornea, lens thickness, an effective lens position, etc. For example, using a patient’s measurements, certain existing system estimate a post-operative manifest refraction in spherical equivalent (MRSE), e.g., for each of a given set of IOL powers available on the market. Using the post-operative MRSEs, the surgeon may then select the IOL power that results in an estimated post-operative MRSE that is closest to the refractive target (i.e., has the lowest estimated post-operative refractive error). However, even with the selected IOL power, the estimated post-operative MRSE may still introduce some post-operative refractive error.

### SUMMARY

Aspects of the present disclosure provide a transposable intraocular lens (IOL). The transposable IOL includes a lens body, including a first lens portion having a first outer surface with a first radius of curvature, a second lens portion having a second outer surface with a second radius of curvature that is different from the first radius of curvature, and a central optic portion between the first lens portion and the second lens portion, and a haptic portion that is coupled to the lens body, the haptic portion configured to support the transposable IOL whether in a first orientation of implantation in a patient’s eye or in a transposed second orientation of implantation in the patient’s eye.

Aspects of the present disclosure also provide a transposable intraocular lens (IOL). The transposable IOL includes a lens body of asymmetric bi-convex shape, having a first outer surface and a second outer surface. The lens body is configured to be positioned with the first outer surface facing a cornea of an eye corresponding to a first predicted refractive error at the corneal plane, and the lens body is configured to be positioned with the second outer surface facing the cornea of the eye corresponding to a second predicted refractive error at the corneal plane.

Aspects of the present disclosure further provide a method for configuring a transposable intraocular lens (IOL). The method includes selecting a target optical power for the transposable IOL, selecting a first target predicted refractive error and a second target predicted refractive error for the transposable IOL, computing a first radius of curvature of a first outer surface of a lens body of the transposable IOL and a second radius of curvature of a second outer surface of the lens body of the transposable IOL based on the target optical power, the first target predicted refractive error, and the second target predicted refractive error, and forming the lens body for the transposable IOL based on the computed first radius of curvature and second radius of curvature.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is noted, however, that the appended drawings illustrate only some aspects of this disclosure and the disclosure may admit to other equally effective embodiments.

FIGS. 1A, 1B, and 1C illustrate a top view, a side view, and another side view, respectively, of an example intraocular lens (IOL).

FIG. 2 is a schematic view of a model eye having a transposable IOL implanted within, according to certain aspects.

FIG. 3A is an enlarged view of a portion of the model eye of FIG. 2 having the IOL implanted with a first orientation, according to certain aspects.

FIG. 3B is an enlarged view of a portion of the model eye of FIG. 2 having the IOL implanted with a second orientation, according to certain aspects.

FIG. 4 depicts an example system for designing, configuring, and/or forming an IOL that is transposable, according to certain aspects.

FIG. 5 depicts example operations for forming an IOL that is transposable, according to certain aspects.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The present disclosure provides a transposable intraocular lens (IOL) with transposable optical properties. As discussed in more detail below, such transposable optical properties may include different refractive outcomes, different spherical aberrations or asphericity, and different toricity. The transposable optical properties can be achieved by the transposable IOL design and the orientation with which transposable IOL is implanted in a patient’s eye.

As mentioned above, cataract surgery may be performed by a surgeon to remove a natural lens from a patient’s eye and replace it with a suitable IOL. For an IOL to achieve the desired refractive outcome for the patient, the surgeon selects the type and power of the IOL based on the patient’s measurements (e.g., pre- or intra-operative measurements). The optical power of an IOL is generally measured in diopters and may be defined at the implant plane inside the eye, although the effective optical value at the corneal plane may be smaller. Typically, optical powers of IOLs are provided in half-diopter spherical equivalent steps over most, if not all, the dioptric power range.

Thus, based on a patient’s pre-operative measurements, a cataract surgeon may select an IOL with an optical power that results in a post-operative spherical equivalent closest to the desired refractive outcome, i.e., an IOL power that has the lowest estimated post-operative refractive error. In some cases, the surgeon may select the appropriate lens from a set of lenses that cover the dioptric power range. However, at least in some cases, as discussed in further detail below, with a set of lenses in half-diopter steps, the IOL that may result in the post-operative spherical equivalent closest to the desired refractive outcome may provide a refractive error that is either slightly myopic or slightly hyperopic.

Accordingly, more resolution in the optical power of IOLs is desired. For example, as advances in pre-operative eye measurements and IOL power calculations improve the determination of the desired corrective strength of the IOL, smaller step sizes in lenses allows a surgeon to select an IOL that provides an optical power closer to the actual desired corrective strength. With smaller step sizes, however, the number of lenses needed to provide a set of lenses that covers the dioptric power range increases. For example, using one-fourth diopter steps sizes, would double the amount of lenses needed for one-half diopter step size lenses to cover the same dioptric power range.

Similarly, it is desirable to more closely match the spherical aberration correction of the IOL to the patient’s eye. Spherical aberration in the human eye is a combination of a positive spherical aberration of the cornea and a negative spherical aberration of the crystalline lens. In young eyes, the positive spherical aberration of the cornea is compensated by the negative spherical aberration of the lens; as a result, overall spherical aberration in the young eye is low. As the eye ages, however, the optical properties of the crystalline lens change, resulting in overall positive spherical aberration and decreased optical performance. In an example use of an aspheric IOL design, the aspheric IOL compensates for the positive spherical aberration of the cornea. For a surgeon, a set of lenses that provides additional options of aspheric IOL designs, allows the surgeon to better match the IOL to the patient’s eye. However, additional lenses leads to increased inventory needs, more manufacturing, and higher costs.

It is also desirable to have lenses that provide a range of toricities. Toric IOLs are often used to correct corneal astigmatisms in cataract surgery. However, similar to the problems described above, providing a set of lenses with additional options of toricity increases the total number of lenses.

Accordingly, aspects of the present disclosure provides a transposable IOL design. With a transposable IOL design, an IOL can be implanted in a patient’s eye with two orientations, a first orientation or a second orientation. Implanting the IOL in the eye with a first orientation (e.g., anterior or posterior facing) achieves a first desired optical power (e.g., a selected first diopter value), a first desired refractive outcome (e.g., a first diopter value of refractive error), a first toricity, and/or a first aspherical design. Implanting the IOL in the patient’s eye with a second orientation (e.g., posterior or anterior facing) achieves a second desired optical power, a second refractive outcome (e.g., a second diopter value of refractive error that is a desired step size from the first diopter value), a second toricity, and/or a second aspherical design. Thus, with a transposable IOL, the number of lenses needed to provide the same range of refractive outcomes, diopter values, toricities, and/or aspherical IOL designs is reduced by a factor of two.

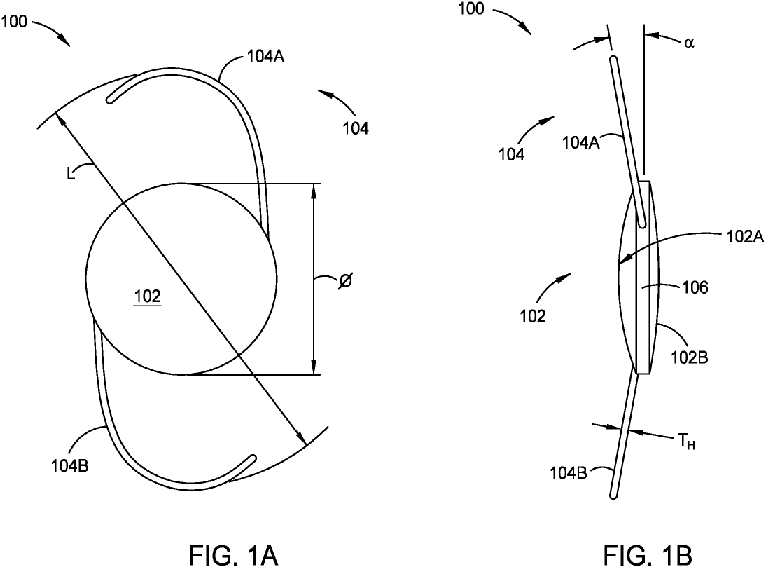

FIGS. 1A, 1B, and 1C illustrate a top view, a side view, and another side view, respectively, of an IOL 100, according to certain aspects. It is noted that the shape and curvatures of IOL 100 are shown for illustrative purposes only and that other shapes and curvatures are also within the scope of this disclosure. IOL 100 includes a lens body 102 and a haptic portion 104 that is coupled to lens body 102.

Lens body 102 includes a first lens portion 102A having a first outer surface with a radius of curvature R₁. Lens body 102 includes a second lens portion 102B having a second outer surface with a radius of curvature R₂. As discussed in more detail below and shown in FIG. 1C, the radii of curvatures, R₁ and R₂, are different for a transposable IOL to provide two different optical powers for IOL 100, based on the orientation with which IOL 100 is implanted in the patient’s eye. For example, R₁ and R2may be formed such that IOL 100 provides two different optical powers that are separate by a desired diopter step-size.

Lens body 102 includes a central optic portion 106 between lens portions 102A and 102B. Lens portions 102A and 102B may be bonded together in a peripheral non-optic portion of lens body 102. Lens body 102 has a diameter Φ. In some examples, the diameter is between about 4.5 mm and about 7.5 mm, for example, about 6.0 mm.

Central optic portion 106 is a transparent optic element of IOL 100 that focuses light on the retina. In some examples, central optic portion 106, first lens portion 102A, and second lens portion 102B are fabricated of a transparent, flexible material, such as a silicone polymeric material, acrylic polymeric material, hydrogel polymeric material or the like. The material may allow IOL 100 to be rolled or folded for introduction into the eye through a small incision. In one example, lens body 102 comprises ultra-violet and blue light absorbing acrylate/methacrylate copolymer. An outer surface of first lens portion 102A and/or of second lens portion 102B may be fabricated of a biocompatible material stiffer than the material of central optic portion 106, such as polymethyl methacrylate (PMMA). Thus, the anterior and posterior outer surfaces of lens portions 102A and 102B can be formed of different materials, such as silicone and PMMA. Lens body 102, depending on the material, can be injection-molded, fabricated with casting techniques, turned by a lathe, etc.

In the example shown in FIG. 1, central optic portion 106 has a bi-convex shape. In other examples, central optic portion 106 may have a plano-convex shape, a convexo-concave shape, or a plano-concave shape. Lens body 102 may have multiple concentric powers for a multi-focal lens design.

Haptic portion 104 includes radially-extending struts (also referred to as “haptics”) 104A and 104B. Haptics 104A and 104B may be fabricated of biocompatible material, such as PMMA. Haptics 104A and 104B are coupled (e.g., glued or welded) to the peripheral portion of lens body 102 or molded along with a portion of lens body 102, and thus extend outwardly from lens body 102 to engage the perimeter wall of the capsular sac of the eye to maintain lens body 102 in a desired position in the eye. Haptics 104A and 104B typically have radial-outward ends that define arcuate terminal portions. The terminal portions of haptics 104A and 104B may be separated by a length L of between about 6 mm and about 22 mm, for example, about 13 mm. Haptics 104A and 104B may have a particular length so that the terminal portions create a slight engagement pressure when in contact with the equatorial region of the capsular sac after being implanted.

Haptics 104A and 104B may be planar with lens body 102. For example, in certain embodiments, the angle α is 0° or about 0° such that lateral compression to IOL 100, when implanted, does not cause vaulting towards the anterior surface or the posterior surface of the IOL 100. In some cases where vaulting can be used as an additional or alternative mechanism to modulate refractive input depending on the orientation of IOL 100, haptics 104A and 104B may be angled to the lens body 102. While FIG. 1 illustrates one example configuration of haptics 104A and 104B, any plate haptics or other types of haptics can be used.

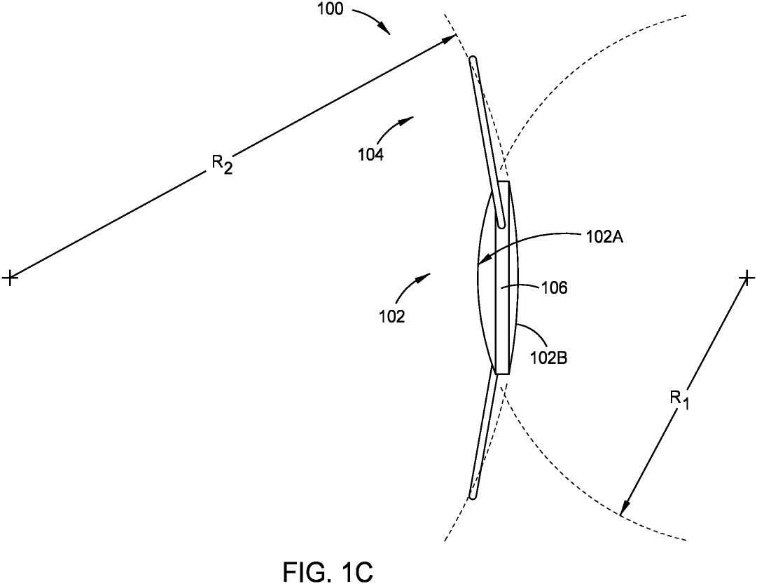

FIG. 2 is a schematic view of a model eye 200 having IOL 100 implanted within, according to certain aspects. In an illustrative example below, IOL 100 has a thickness T_{IOL} (i.e., the distance between anterior outer surface and posterior outer surface of IOL 100 at the middle of IOL 100). Model eye 200 includes cornea 202 having a refractive index n_{cornea} (e.g., in an illustrative example below 1.376) and a thickness T_{cornea}. Cornea 202 has an anterior surface 202A with a radius of curvature R_{A} (e.g., in an illustrative example below 7.80 mm) and a posterior surface 202P with a radius of curvature R_{P} (e.g., in an illustrative example below 6.47 mm). Aqueous humor 206 has a depth T_{A} (i.e., the distance between posterior surface 202P of the cornea 202 to the anterior outer surface of IOL 100) (e.g., in an illustrative example below 4.62 mm). Vitreous humor 208 has a depth T_{V} (i.e., the distance between retina 204 and posterior outer surface of IOL 100) (e.g., in an illustrative example below 18.18 mm). Model eye 200 has an overall axial length ALX= T_{cornea}+T_{A}+ T_{IOL}+T_{V} (i.e., the distance between anterior surface 202A of cornea 202 and retina 204) (e.g., in an illustrative example below 24.05 mm). The example illustrative values for the parameters T_{IOL}, T_{cornea}, T_{A}, T_{V}, R_{A}, R_{P}, and n_{cornea} shown herein, which are used in the following example refractive calculations, are typical for human eyes. However, for true values, accurately measured or predicted values of a specific patient’s eye are used.

Aqueous humor 206 and vitreous humor 208 are both assumed to have a refractive index n_{medium} (e.g., of 1.336). Optical power P of IOL 100 can be calculated as:

$$\begin{matrix}P = \frac{{n}_{\text{IOL}} - {n}_{\text{medium}}}{{R}_{1}} + \frac{{n}_{\text{medium}} - {n}_{\text{IOL}}}{{R}_{2}} - \\ \frac{T}{{n}_{\text{IOL}}} \frac{{n}_{\text{IOL}} - {n}_{\text{medium}}}{{R}_{1}} ⋅ \frac{{n}_{\text{medium}} - {n}_{\text{IOL}}}{{R}_{2}} ,\end{matrix}$$

where n_{IOL} is the refractive index of lens body 102, R₁ is a radius of curvature of the anterior outer surface of lens body 102, R₂ is a radius of curvature of the posterior outer surface of the lens body 102, and T is a thickness of the central optic portion 106 of the lens body 102.

According to matrix ray tracing methods known in the art, a relationship between a light ray entering the eye at anterior surface 202A of cornea 202 and the light ray exiting the eye at the fovea (center of retina 204) can be analytically calculated as:

$$\begin{matrix}\left[\begin{matrix}{y}_{\text{retina}} \\ {α}_{\text{retina}}\end{matrix}\right] \\ = \left[\begin{matrix}1 & {T}_{V} \\ 0 & 1\end{matrix}\right] \left[\begin{matrix}1 & 0 \\ \frac{{n}_{\text{medium}} - {n}_{\text{IOL}}}{{R}_{2} ⋅ {n}_{\text{IOL}}} & \frac{{n}_{\text{medium}}}{{n}_{\text{IOL}}}\end{matrix}\right] \left[\begin{matrix}1 & {T}_{\text{IOL}} \\ 0 & 1\end{matrix}\right] \left[\begin{matrix}1 & 0 \\ - \frac{{n}_{\text{IOL}} - {n}_{\text{medium}}}{{R}_{1} ⋅ {n}_{\text{medium}}} & \frac{{n}_{\text{medium}}}{{n}_{\text{IOL}}}\end{matrix}\right] \\ \left[\begin{matrix}1 & {T}_{A} \\ 0 & 1\end{matrix}\right] \left[\begin{matrix}1 \\ - \frac{{n}_{\text{medium}} -}{{R}_{\text{P}} ⋅ {n}_{\text{co}}}\end{matrix}\right)\end{matrix}$$

$$\begin{matrix}\left[\begin{matrix}{y}_{\text{retina}} \\ {α}_{\text{retina}}\end{matrix}\right] \\ = \left[\begin{matrix}1 & {T}_{V} \\ 0 & 1\end{matrix}\right] \left[\begin{matrix}1 & 0 \\ \frac{{n}_{\text{medium}} - {n}_{\text{IOL}}}{{R}_{2} ⋅ {n}_{\text{IOL}}} & \frac{{n}_{\text{medium}}}{{n}_{\text{IOL}}}\end{matrix}\right] \left[\begin{matrix}1 & {T}_{\text{IOL}} \\ 0 & 1\end{matrix}\right] \left[\begin{matrix}1 & 0 \\ - \frac{{n}_{\text{IOL}} - {n}_{\text{medium}}}{{R}_{1} ⋅ {n}_{\text{medium}}} & \frac{{n}_{\text{medium}}}{{n}_{\text{IOL}}}\end{matrix}\right] \\ \left[\begin{matrix}1 & {T}_{A} \\ 0 & 1\end{matrix}\right] \left[\begin{matrix}1 \\ - \frac{{n}_{\text{medium}} -}{{R}_{\text{P}} ⋅ {n}_{\text{co}}}\end{matrix}\right)\end{matrix}$$

where y_{cornea} is a displacement of the entering light ray at anterior surface 202A of cornea, and α_{cornea} is an angle of the propagation of the entering light ray relative to the optical axis. To calculate the refractive error or the eye it is more intuitive to consider the time reverse propagation of light, i.e., from the retina back out the cornea. The ray matrix equation for the time-reversed situation is:

$$\begin{matrix}\left[\begin{matrix}{y}_{\text{cornea}} \\ {α}_{\text{cornea}}\end{matrix}\right] \\ = \left[\begin{matrix}1 & 0 \\ \frac{{n}_{\text{cornea}} - 1}{{R}_{\text{A}}} & {n}_{\text{cornea}}\end{matrix}\right] \left[\begin{matrix}1 & {T}_{C} \\ 0 & 1\end{matrix}\right] \left[\begin{matrix}1 & 0 \\ - \frac{{n}_{\text{medium}} - {n}_{\text{cornea}}}{{R}_{\text{P}} ⋅ {n}_{\text{cornea}}} & \frac{{n}_{\text{medium}}}{{n}_{\text{cornea}}}\end{matrix}\right] \left[\begin{matrix}1 & {T}_{A} \\ 0 & 1\end{matrix}\right] \\ \left[\begin{matrix}1 & 0 \\ - \frac{{n}_{\text{IOL}} - {n}_{\text{medium}}}{{R}_{1} ⋅ {n}_{\text{medium}}} & \frac{{n}_{\text{med}}}{{n}_{\text{IO}}}\end{matrix}\right)\end{matrix}$$

$$\begin{matrix}\left[\begin{matrix}{y}_{\text{cornea}} \\ {α}_{\text{cornea}}\end{matrix}\right] \\ = \left[\begin{matrix}1 & 0 \\ \frac{{n}_{\text{cornea}} - 1}{{R}_{\text{A}}} & {n}_{\text{cornea}}\end{matrix}\right] \left[\begin{matrix}1 & {T}_{C} \\ 0 & 1\end{matrix}\right] \left[\begin{matrix}1 & 0 \\ - \frac{{n}_{\text{medium}} - {n}_{\text{cornea}}}{{R}_{\text{P}} ⋅ {n}_{\text{cornea}}} & \frac{{n}_{\text{medium}}}{{n}_{\text{cornea}}}\end{matrix}\right] \left[\begin{matrix}1 & {T}_{A} \\ 0 & 1\end{matrix}\right] \\ \left[\begin{matrix}1 & 0 \\ - \frac{{n}_{\text{IOL}} - {n}_{\text{medium}}}{{R}_{1} ⋅ {n}_{\text{medium}}} & \frac{{n}_{\text{med}}}{{n}_{\text{IO}}}\end{matrix}\right)\end{matrix}$$

A ray originating at the fovea has zero retinal displacement, i.e., yᵣₑₜᵢₙₐ = 0. If we set the retinal ray angle to some non-zero value (e.g., 0.01), the above equation will yield parameters describing the corresponding ray exiting the cornea. The refractive error Rₓ can be calculated as a ratio of a displacement y_{cornea} of the exiting light ray at retina 204 from the optical axis and an angle α_{cornea} of the propagation of the exiting light ray relative to the optical axis (i.e., Rₓ = αᵣₑₜᵢₙₐ/yᵣₑₜᵢₙₐ).

As described above, a typical set of IOLs includes IOLs having optical powers with a one-half diopter step size, which can lead to a refractive outcome that is either myopic or hyperopic. For example, according to the optical power formula above, an existing IOL having a lens body of a symmetric bi-convex shape (i.e., R₁ = R₂) provides power P = 21 diopters (D), assuming the radii of curvature is R₁ = R₂ = 20.33 mm, the thickness of central optic portion of lens body 102 is T = 0.7 mm, and the refractive index of lens body 102 is n_{IOL} = 1.55. According to the ray tracing methods described above along with the above illustrative anatomical values for the parameters T_{IOL}, T_{cornea}, T_{A}, T_{V}, R_{A}, R_{P}, and n_{cornea}, the IOL is predicted to have a refractive error of +0.15 D at the cornea plane in the eye (i.e., slightly hyperopic).

If an IOL is used instead with the radii of curvature R₁ = R₂ = 19.86 mm, with the other parameters described above remaining the same, the IOL provides optical power P = 21.5 D, a one-half diopter step size increase with respect to the IOL with the 20.33 mm radius of curvature. According to the paraxial model, this configuration is predicted to have a refractive error of -0.19 D at the corneal plane in the eye (i.e., slightly myopic).

Thus, with a one-half diopter step-size, a cataract surgeon, in this example, is forced to select between an IOL having the optical power P = 21 D, which results in a slightly hyperopic outcome, and an IOL having the optical power P= 21.5 D, which results in a slightly myopic outcome. The offset in the refractive errors, 0.34 D, is typical for two IOLs 100 having powers with a 0.5 D step size. Accordingly, using transposable IOLs allows for providing more resolution in the refractive outcomes offered by a set of IOLs, thereby providing more options for a cataract surgeon to reduce the post-operative refractive error.

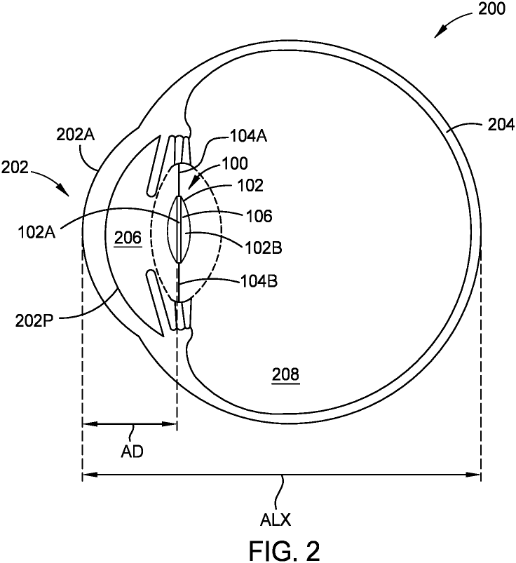

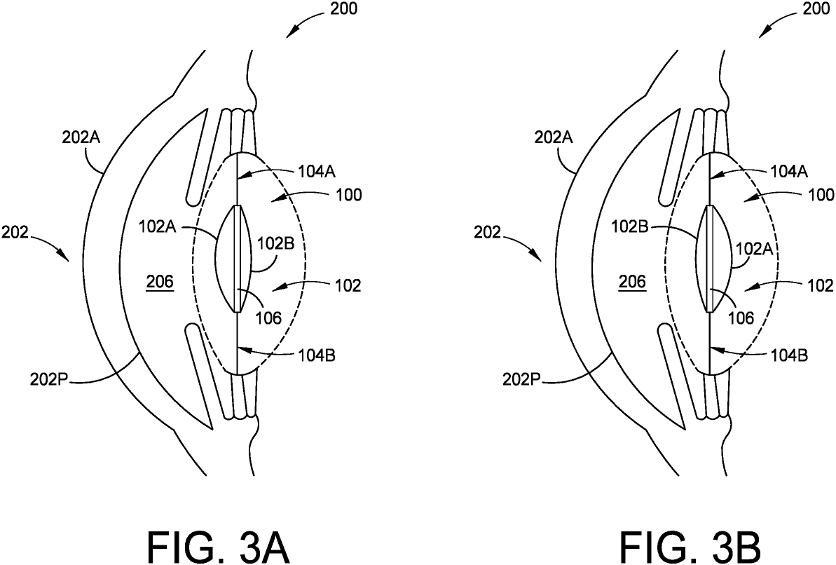

FIGS. 3A and 3B are enlarged views of a portion of a model eye 200 having the transposable asymmetric IOL 100 implanted within, according to certain aspects. As depicted in FIGS. 3A and 3B, lens body 102 of the IOL 100 is of asymmetric bi-convex shape (i.e., R₁ ≠ R₂). In the example of FIG. 3A, the first lens portion 102A faces the cornea 202 of model eye 200 and the second lens portion 102B faces the retina 204 (shown in FIG. 2). As shown, a radius of curvature R₁ of the outer surface of the first lens portion 102A is different from a radius of curvature R₂ of the outer surface of the second lens portion 102B. In FIG. 3B, the IOL 100 shown in FIG. 3A is transposed such that the first lens portion 102A faces retina 204 and second lens portion 102B faces cornea 202.

Returning to the illustrative example discussed above, the asymmetric bi-convex shape IOL 100 may have (e.g., instead of R₁ = R₂ = 19.86 mm or 20.33 mm) a first radius of curvature R₁ = 16.75 mm and a second radius of curvature R₂ = 25.88 mm. With this IOL design, IOL 100 provides an optical power P = 21 D, but the refractive outcome depends on the orientation that IOL 100 is implanted. According to the paraxial model along with the above example anatomical values for the parameters T_{IOL}, T_{cornea}, T_{A}, T_{V}, R_{A}, R_{P}, and n_{cornea}, IOL 100 is predicted to have a refractive error of 0.07 D when the IOL 100 is positioned as shown in FIG. 3A with first lens portion 102A facing cornea 202. The same IOL 100, when transposed as shown in FIG. 3B, is expected to have a refractive error of 0.24 D when second lens portion 102B is facing cornea 202. The offset in the refractive error between the IOL 100 positioned as shown in FIG. 3A and the same IOL 100 transposed as shown in FIG. 3B is reduced to 0.17D from the offset of about 0.34 D between two symmetric biconvex IOLs having powers with a 0.5 D step size.

Thus, transposable IOLs having lens bodies with asymmetric bi-convex shapes provide additional treatment options with reduced refractive errors. In some embodiments, one of the radii of curvatures, R₁ and R₂ is determined by a desired (i.e., target) IOL power, and the other of the radii of curvatures, R₁ and R₂ is adjusted accordingly to provide a desired change in the refractive error when IOL 100 is transposed. In some other embodiments, the radii of curvatures, R₁ and R₂ are determined such that the overall mass of IOL 100 low, which would facilitate implantation through smaller surgical incisions. For example, a cataract surgeon can have a set of IOLs with more resolution in the refractive outcomes, e.g., using a fraction of the number of transposable IOLs than would be needed for typical non-transposable IOLs. In addition, for a given patient, based on the pre-operative measurements of the patient’s eye, two different predicted post-operative refractive outcomes corresponding to the two different orientations of implantation in the patient’s eye can be calculated for each transposable IOL. This allows the surgeon to not only select the IOL, but also the implantation orientation of the selected IOL that provides the lowest predicted post-operative outcome.

In certain embodiments, transposable asymmetric IOL 100 may be toric in design. For example, IOL 100 may be designed with two different toricities. A toric transposable asymmetric IOL can be used in modulated astigmatism treatment, for example. For toric IOLs, the transposition may have a minor effect on the net cylinder correction.

According to certain aspects, a transposable asymmetric IOL is provided that has different asphericities. For example, IOL 100 can have a first set of surface aberrations for first lens portion 102A providing a first asphericity and second set of surface aberrations, different than the first set of surface aberrations, for second lens portion 102B providing a second asphericity. Asphericity can be used for spherical aberration compensation. With a transposable IOL that provides two different asphericities, a cataract surgeon has twice the number of options for each IOL.

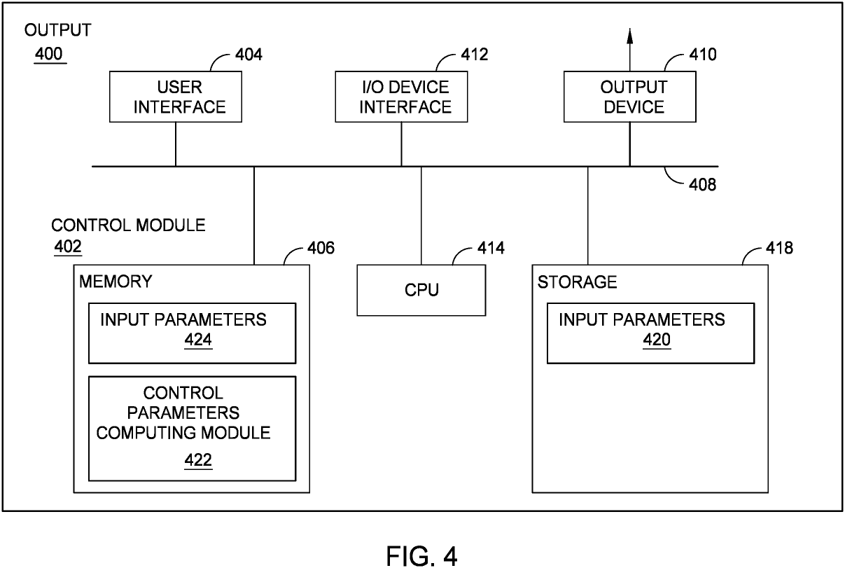

FIG. 4 depicts an example system 400 for designing, configuring, and/or forming an IOL 100 that is transposable, according to certain aspects of the disclosure. As shown, system 400 includes, but is not limited to, a control module 402, a user interface display 404, an interconnect 408, an output device 410, and at least one I/O device interface 412, which may allow for the connection of various I/O devices (e.g., keyboards, displays, mouse devices, pen input, etc.) to system 400.

Control module 402 includes a central processing unit (CPU) 414, a memory 416, and a storage 418. CPU 414 may retrieve and execute programming instructions stored in memory 416. Similarly, CPU 414 may retrieve and store application data residing in memory 416. Interconnect 408 transmits data, among CPU 414, I/O device interface 412, user interface display 404, memory 416, storage 418, output device 410, etc. CPU 414 can represent a single CPU, multiple CPUs, a single CPU having multiple processing cores, and the like. Additionally, in certain aspects, memory 416 represents a random access memory. Furthermore, in certain aspects, storage 418 may be a disk drive. Although shown as a single unit, storage 418 may be a combination of fixed or removable storage devices, such as fixed disc drives, removable memory cards or optical storage, network attached storage (NAS), or a storage area-network (SAN).

As shown, storage 418 includes input parameters 420. Input parameters 420 include example anatomic parameters of a model eye (e.g., average values) and a desired range of predicted refractive outcomes at the cornea, in order to generate output radii of curvature that can be used to form an IOL or set of IOLs that provides the desired range of predicted refractive outcomes. For example, input parameters 420 may include a refractive index n_{cornea} of a cornea, a radius of curvature R_{A} of the anterior surface of the cornea, a radius of curvature R_{P} of the posterior surface of the cornea, an overall axial length ALX of an eye, a depth T_{A} of the aqueous humor, a depth T_{V} of the vitreous humor, a desired IOL power P, a first desired predicted refractive error, and a second desired predicted refractive error. Memory 416 includes a computing module 422 for computing a first radius of curvature R₁ and a second radius of curvature R₂ that provide the desired IOL power P and refractive errors at the corneal plane. In addition, memory 416 includes input parameters 424.

In certain aspects, input parameters 424 correspond to input parameters 420 or at least a subset thereof. During the computation of the radii of curvature R₁ and R₂, the input parameters 424 are retrieved from storage 418 and executed in memory 416. In such an example, computing module 422 comprises executable instructions (e.g., including one or more of the formulas described herein) for computing the radii of curvature R₁ and R₂ based on the input parameters 424. In certain other aspects, input parameters 424 correspond to parameters received from a user through user interface display 404. In such aspects, computing module 422 comprises executable instructions for computing the radii of curvature R₁ and R₂ based on information received from user interface display 404.

In certain aspects, the computed radii of curvature R₁ and R₂ are output via output device 410 to a lens manufacturing system that is configured to receive the control parameters and form a lens accordingly. In certain other aspects, system 400 itself is representative of at least a part of a lens manufacturing systems. In such aspects, control module 402 then causes hardware components (not shown) of system 400 to form the lens according to the control parameters. The details and operations of a lens manufacturing system are known to one of ordinary skill in the art and are omitted here for brevity.

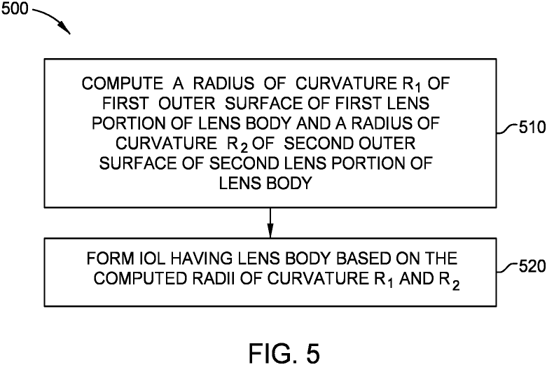

FIG. 5 depicts example operations 500 for forming an IOL 100 that is transposable. In some aspects, operation 510 of operations 500 is performed by one system (e.g., system 400) and operation 520 is performed by a lens manufacturing system. In some other aspects, both of operations 510 and 520 are performed by system 400 or the lens manufacturing system.

At operation 510, a first radius of curvature R₁ of the outer surface of the first lens portion 102A of the lens body and a second radius of curvature R₂ of the outer surface of the second lens portion 102B of the lens body 102 are computed based on input parameters (i.e., T_{IOL}, T_{cornea}, T₄, T_{V,} n_{cornea}, R_{A}, R_{P}, ALX, desired IOL power P, and first desired predicted refractive error, and second desired predicted refractive error). The computations performed at operation 510 are based on one or more of the embodiments, including the formulas, described herein.

At operation 520, an IOL 100 having a lens body 102 based on the computed radii of curvature R₁ and R₂ and a haptic portion 104 coupled to the lens body 102 is formed, using appropriate methods, systems, and devices typically used for manufacturing lenses.

The aspects described herein provide IOLs that can be transposable to provide two options for optical outcomes, such as optical power, refractive error, toricity, and/or asphericity, depending on the orientation of the IOL relative to the cornea of the eye, and thus, provide increased refractive accuracy. Increasing refractive accuracy reduces the need for specialized post-operative equipment and/or patient return visits for adjustments or corrections.

The aspects herein may be applied to any type of IOL, including monofocal, multifocal, and extended depth of focus IOL surface features. Providing transposable IOLs doubles the number of optical treatment options per IOL, allowing for a family of lenses with higher resolution in refractive error, asphericity, or toricity, while reducing the total number of lenses needed in the family of lenses.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form. A phrase referring to “at least one of” a list of items refers to any combination of those items, including single members and duplicate members. As an example, “at least one of: a, b, or c” is intended to cover, for example: a, b, c, a-b, a-c, b-c, a-b-c, aa, a-bb, a-b-cc, and etc.

While the foregoing is directed to embodiments of the present disclosure, other and further embodiments of the disclosure may be devised without departing from the basic scope thereof, and the scope thereof is determined by the claims that follow.

1. A transposable intraocular lens (IOL), comprising:
a lens body, including:
a first lens portion having a first outer surface with a first radius of curvature;
a second lens portion having a second outer surface with a second radius of curvature that is different from the first radius of curvature; and
a central optic portion between the first lens portion and the second lens portion; and
a haptic portion that is coupled to the lens body, the haptic portion configured to support the transposable IOL whether in a first orientation of implantation in a patient’s eye or in a transposed second orientation of implantation in the patient’s eye.

2. The transposable IOL of claim 1, wherein the haptic portion is planar with the lens body.

3. The transposable IOL of claim 1, wherein:
the first radius of curvature is determined by a target IOL power, and
the second radius of curvature is determined by a target change in a refractive error when the lens body is transposed.

4. The transposable IOL of claim 1, wherein the lens body comprises acrylate/methacrylate copolymer.

5. The transposable IOL of claim 1, wherein the lens body has a diameter of between 4.5 mm and 7.5 mm.

6. The transposable IOL of claim 1, wherein the haptic portion comprises polymethyl methacrylate (PMMA).

7. The transposable IOL of claim 1, wherein:
the haptic portion comprises two radially-extending struts that extend outwardly from the lens body, and
terminal ends of the two radially-extending struts are separated by a distance of between 8 mm and 13 mm.

8. A transposable intraocular lens (IOL), comprising:
a lens body of asymmetric bi-convex shape, having a first outer surface and a second outer surface, wherein:
the lens body is configured to be positioned with the first outer surface facing a cornea of an eye corresponding to a first predicted refractive error at the corneal plane; and
the lens body is configured to be positioned with the second outer surface facing the cornea of the eye corresponding to a second predicted refractive error at the corneal plane.

9. The transposable IOL of claim 8, wherein a difference between the first predicted refractive error and the second predicted refractive error is less than 0.34 diopters.

10. The transposable IOL of claim 8, wherein:
a first radius of curvature of the first outer surface is determined by a target IOL power, and
a second radius of curvature of the second outer surface is determined by a target change in a refractive error when the lens body is transposed.

11_{.}. The transposable IOL of claim 8, wherein the lens body comprises acrylate/methacrylate copolymer.

12. The transposable IOL of claim 8, wherein the lens body has a diameter of between 4.5 mm and 7.5 mm.

13. The transposable IOL of claim 8, further comprising:
a haptic portion that is coupled to the lens body, wherein the haptic portion is planar with the lens body.

14. The transposable IOL of claim 13, wherein the haptic portion comprises polymethyl methacrylate (PMMA).

15. The transposable IOL of claim 13, wherein:
the haptic portion comprises two radially-extending struts that extend outwardly from the lens body, and
terminal ends of the two radially-extending struts are separated by a distance of between 8 mm and 13 mm.

16. A method for configuring a transposable intraocular lens (IOL), comprising:
selecting a target optical power for the transposable IOL;
selecting a first target predicted refractive error and a second target predicted refractive error for the transposable IOL;
computing a first radius of curvature of a first outer surface of a lens body of the transposable IOL and a second radius of curvature of a second outer surface of the lens body of the transposable IOL based on the target optical power, the first target predicted refractive error, and the second target predicted refractive error; and
forming the lens body for the transposable IOL based on the computed first radius of curvature and second radius of curvature.

17. The method of claim 16, wherein:
the lens body is configured to provide the first target predicted refractive error when the transposable IOL is implanted in a patient’s eye with the first outer surface facing the patient’s cornea,
the computing is based on one or more of: a refractive index of a cornea, a radius of curvature of an anterior surface of the cornea, a radius of curvature of a posterior surface of the cornea, an overall axial length of the eye, a first depth of an aqueous corresponding to when the transposable IOL is implanted in the patient’s eye with the first outer surface facing the patient’s cornea, and a first depth of a vitreous corresponding to when the transposable IOL is implanted in the patient’s eye with the first outer surface facing the patient’s cornea; and
the lens body is configured to provide the second target predicted refractive error when the transposable IOL is implanted in the patient’s eye with the second outer surface facing the patient’s cornea, and
the computing is based on one or more of: the refractive index of the cornea, the radius of curvature of the anterior surface of the cornea, the radius of curvature of the posterior surface of the cornea, the overall axial length of the eye, a second depth of the aqueous corresponding to when the transposable IOL is implanted in the patient’s eye with the second outer surface facing the patient’s cornea, and a second depth of the vitreous corresponding to when the transposable IOL is implanted in the patient’s eye with the second outer surface facing the patient’s cornea.

18. The method of claim 16, further comprising:
attaching a haptic portion to the lens body, the haptic portion configured to support the transposable IOL whether in a first orientation of implantation in a patient’s eye or in a transposed second orientation of implantation in the patient’s eye.

19. The method of claim 18, wherein the haptic portion is planar with the lens body.

20. The method of claim 18, wherein
the lens body comprises acrylate/methacrylate copolymer, and
the haptic portion comprises polymethyl methacrylate (PMMA).
